(19) United States
(12) Patent Application Publication
(10) Pub. No.: US 2025/0367185 A1
(43) Pub. Date: Dec. 4, 2025

(54) TRANSLESION SYNTHESIS POLYMERASE INHIBITORS AND USES THEREOF

(71) Applicant: THE UNIVERSITY OF VERMONT AND STATE AGRICULTURAL COLLEGE, Burlington, VT (US)

(72) Inventor: Nimrat Chatterjee, South Burlington, VT (US)

(73) Assignee: THE UNIVERSITY OF VERMONT AND STATE AGRICULTURAL COLLEGE, Burlington, VT (US)

(21) Appl. No.: 19/107,830

(22) PCT Filed: Aug. 31, 2023
(86) PCT No.: PCT/US2023/031587

§ 371 Date: Feb. 28, 2025

Related U.S. Application Data
(60) Provisional application No. 63/403,240, filed on Sep. 1, 2022.

(51) Int. Cl. A61K 31/47; A61P 31/14

(52) U.S. Cl. CPC A61K 31/47; A61P 31/14

(57) ABSTRACT

Disclosed herein are compositions and methods relating to the inhibitors of the translesion synthesis (TLS) pathway, including methods of preventing or treating RNA viruses and prolonged symptoms (for example, long COVID).

### RELATED APPLICATION

This application claims the benefit under 35 U.S.C. § 119 (e) of U.S. provisional application No. 63/403,240, filed Sep. 1, 2022, which is herein incorporated by reference in its entirety.

### BACKGROUND OF THE INVENTION

The 2020 outbreak of novel coronavirus disease 2019 (COVID-19) infections is associated with a high mortality rate death toll. Coronavirus disease 2019 (COVID-19), an infectious disease caused by severe acute respiratory syndrome coronavirus 2 (SARS-CoV-2), is an RNA virus that has spread to six continents, rapidly infecting millions of people worldwide. The long-term effects of certain COVID-19 infections, termed long-COVID, have emerged. Treatments for COVID-19 have targeted to the amelioration of symptoms during the initial infection.

### SUMMARY OF THE INVENTION

In some aspects, the disclosure relates to a method of treating a viral infection in a subject, wherein the method comprises administering to the subject an inhibitor of the translesion synthesis (TLS) pathway.

In some embodiments, the inhibitor comprises an inhibitor of a mutagenic translesion synthesis polymerase. In some embodiments, the mutagenic translesion synthesis polymerase is selected from the group consisting of: POLh, POLK, POLi, REV1, REV3L, and REV7. In some embodiments, the mutagenic translesion synthesis polymerase comprises REV1.

In some embodiments, the inhibitor of a translesion synthesis polymerase comprises a small molecule inhibitor. In some embodiments, the small molecule inhibitor comprises Compound 1:

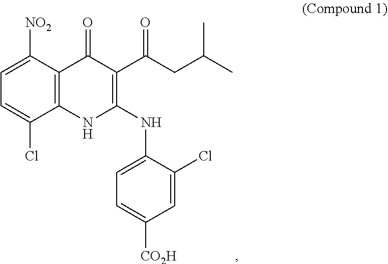 [SMILES: CC(C)CC(=O)c1c(Nc2ccc(C(=O)O)cc2Cl)[nH]c2c(Cl)ccc([N+](=O)[O-])c2c1=O]

In some embodiments, the viral infection is caused by an RNA virus. In some embodiments, the RNA virus is selected from the group consisting of: coronavirus, Dengue virus, retrovirus, flavivirus, Nipah virus, West Nile virus, human papillomavirus, respiratory syncytial virus, filovirus, Zaire ebolavirus, Sudan ebolavirus, Marburg virus, and influenza virus. In some embodiments, the viral infection is a coronavirus. In some embodiments, the coronavirus comprises a betacoronavirus. In some embodiments, the betacoronavirus comprises SARS-COV-2 or a variant thereof. In some embodiments, the SARS-COV-2 variant is selected from the group consisting of: alpha, beta, gamma, delta, omicron BA-1, omicron BA-2, omicron BA.4, omicron BA.5, XBB.1.5, XBB.1.16, and EG.5. In some embodiments, the variant comprises a circulating SARS-COV-2 variant.

In some embodiments, the subject has, or is suspected of having, long COVID. In some embodiments, the subject is human. In some embodiments, the subject has at least one symptom of long COVID. In some embodiments, the at least one symptom of long COVID is selected from the group consisting of: cardiomyopathy, neurological issues, diabetes, respiratory system disorders, nervous system and neurocognitive disorders, mental health disorders, metabolic disorders, gastrointestinal disorders, musculoskeletal pain, anemia, headaches, shortness of breath, anosmia, parosmia, muscle weakness, and low fever.

In some embodiments, the subject is administered the inhibitor by oral administration or intravenous administration.

The disclosure, in another aspect, provides a pharmaceutical composition suitable for treating a viral infection in a subject, wherein the pharmaceutical composition comprises an inhibitor of the translesion synthesis (TLS) pathway.

In some embodiments, the pharmaceutical composition further comprises a pharmaceutically acceptable carrier.

In some embodiments, the inhibitor comprises an inhibitor of a mutagenic translesion synthesis polymerase. In some embodiments, the mutagenic translesion synthesis polymerase is selected from the group consisting of: POLh, POLK, POLi, REV1, REV3L, and REV7. In some embodiments, the mutagenic translesion synthesis polymerase comprises REV1.

In some embodiments, the inhibitor of a translesion synthesis polymerase comprises a small molecule inhibitor. In some embodiments, the small molecule inhibitor comprises Compound 1.

In some embodiments, the viral infection is caused by an RNA virus. In some embodiments, the RNA virus is selected from the group consisting of: coronavirus, Dengue virus, retrovirus, flavivirus, Nipah virus, West Nile virus, human papillomavirus, respiratory syncytial virus, filovirus, Zaire ebolavirus, Sudan ebolavirus, Marburg virus, and influenza virus. In some embodiments, the viral infection is a coronavirus. In some embodiments, the coronavirus comprises a betacoronavirus. In some embodiments, the betacoronavirus comprises SARS-COV-2 or a variant thereof. In some embodiments, the SARS-COV-2 variant is selected from the group consisting of: alpha, beta, gamma, delta, omicron BA-1, omicron BA-2, omicron BA.4, omicron BA.5, XBB.1.5, XBB.1.16, and EG.5. In some embodiments, the variant comprises a circulating SARS-COV-2 variant.

In some embodiments, the subject has, or is suspected of having, long COVID. In some embodiments, the subject is human. In some embodiments, the subject has at least one symptom of long COVID. In some embodiments, the at least one symptom of long COVID is selected from the group consisting of: cardiomyopathy, neurological issues, diabetes, respiratory system disorders, nervous system and neurocognitive disorders, mental health disorders, metabolic disorders, gastrointestinal disorders, musculoskeletal pain, anemia, headaches, shortness of breath, anosmia, parosmia, muscle weakness, and low fever (e.g., long COVID symptoms).

In some embodiments, the subject is administered the inhibitor by oral administration or intravenous administration.

In some embodiments, the pharmaceutical composition is formulated as a capsule. In some embodiments, the pharmaceutical composition is acceptable for oral administration.

In some embodiments, the pharmaceutical composition is administered according to a dosing schedule sufficient to alleviate the at least one long-COVID symptom.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a graph showing the relative reduction of mRNA in A549-ACE2+ cells post treatment with 10 μM Compound 1 (“JH”) at 48 hours post-infection with COVID-19.

FIG. 2 is a graph showing the relative reduction of mRNA in untreated A549-ACE2+ cells (“Not Treated”) and in cells post-treatment with Compound 1 (“JH Treated”) at 24 and 48 hours post-infection with Dengue virus.

FIG. 3 is a graph showing the relative reduction of mRNA in untreated A549-ACE2+ cells and cells post treatment with Compound 1 (“JH Treated”) or untreated (“Not Treated”) 5 days post-infection with Dengue virus.

FIG. 4 is a graph showing the reduction in the percentage of nucleocapsid positive cells infected with variants of SARS-COV-2 post treatment with Compound 1.

### DETAILED DESCRIPTION OF THE INVENTION

Since the beginning of the 21st century, three coronaviruses: severe acute respiratory syndrome coronavirus 1 (SARS-COV-1), Middle Eastern respiratory syndrome coronavirus (MERS-COV), and SARS-COV-2, have undergone zoonotic transmission to trigger fatal pneumonia in humans. As an example, SARS-COV-2-induced COVID-19 presents with symptoms of acute lung injury, subsequent acute respiratory distress syndrome, and, in some cases, results in prolonged health effects such as long COVID. Long COVID develops even after a full regimen of vaccinations and boosters.

Despite the rapid development and implementation of vaccines against COVID-19, long COVID can develop in vaccinated individuals that presented with mild symptoms during infection. It is estimated that more than half of infected people experience long COVID. Long COVID has been associated with symptoms such as cardiomyopathy, neurological issues, respiratory system disorders, nervous system and neurocognitive disorders, mental health disorders, metabolic disorders, gastrointestinal disorders, and muscle weakness; however, treatment against the long-term effects of COVID-19 infection are limited.

Disclosed herein are compositions and methods of preventing and treating RNA viruses (e.g., SARS-COV-2) and their long-term effects (e.g., long COVID). As is demonstrated below, it was found that SARS-COV-2 infection triggers host cell genome instability by modulating expression of DNA repair and mutagenic translesion synthesis (TLS) molecules, leading to increased mutagenesis, telomere dysregulation, and elevated microsatellite instability (MSI). Thus, described herein are TLS pathway inhibitors (e.g., TLS polymerase inhibitors), which suppress viral proliferation and repress viral-dependent genomic instability. In this way, the compositions and methods provided herein may be used, in some embodiments, to prevent and/or treat RNA viruses and their long-term effects (e.g., long COVID).

### Translesion Synthesis Polymerase Inhibitors

Provided herein, in some embodiments, are translesion synthesis (TLS) pathway inhibitors. Translesion synthesis takes place in two steps in mammalian cells: first, a nucleotide is inserted opposite to a lesion with an insertion TLS DNA polymerase (e.g., POL k, POL i, POL h, or REV1), and second, elongation of the resulting terminus is performed with an extension TLS DNA polymerase. Translesion synthesis (TLS) polymerases, therefore, are error-prone enzymes that facilitate DNA replication in the presence of DNA damage; however, many have error rates exceeding 1 in 1000. TLS polymerases are capable of bypassing DNA lesions which are implicated in meiotic double-strand break repair. There are over a dozen described TLS polymerases in human cells with increased expression leading to hypermutation. Without wishing to be bound by theory, it is thought that inhibiting the TLS pathway reduces the deleterious consequences of viral infections (e.g., by maintaining host cell genome stability). Examples of inhibitors of the TLS pathway include those described in WO 2020/077014, the entire contents of which are incorporated herein in their entirety.

In some embodiments, the TLS pathway inhibitor comprises an inhibitor of an TLS polymerase (e.g., a mutagenic translesion synthesis polymerase). In some embodiments, the TLS polymerase is an insertion TLS DNA polymerase or an extension TLS DNA polymerase.

In some embodiments, the insertion TLS DNA polymerase is selected from the group consisting of: POLh, POLK, POLi, and REV1. In some embodiments, the TLS pathway inhibitor comprises an inhibitor of REV1. REV1 is a scaffolding protein that recruits other translesion DNA polymerases to DNA lesions (UniProt Accession No. Q9UBZ9). A deoxycytidyl transferase involved in DNA repair, REV1 transfers a dCMP residue from dCTP to the 3′-end of a DNA primer in a template-dependent reaction and may assist in the first step in the bypass of abasic lesions by the insertion of a nucleotide opposite the lesion.

In some embodiments, the extension TLS DNA polymerase comprises a component of the DNA polymerase delta complex (e.g., POLD1, POLD2, POLD3, and POLD4) or the DNA polymerase zeta complex (e.g., B-family polymerase complex POL ζ (e.g., REV3L and REV7).

Inhibitors of the TLS pathway include, but are not limited to small molecules, antibodies, antibody derivatives (including Fab fragments and scFvs), antibody drug complexes, antisense oligonucleotides, siRNAs, aptamers, peptides, and pseudopeptides. A TLS pathway inhibitor, in some embodiments, refers to a compound that reduces the level of expression of any one of the components of the TLS pathway (e.g., an insertion TLS DNA polymerase or an extension TLS DNA polymerase) relative to a baseline level of expression (e.g., a level prior to treatment with the compound). In some embodiments, the inhibitor inhibits TLS pathway activity by greater than 10%, 33%, 50%, 90%, 95% or 99% as compared to a TLS pathway that is not inhibited by a method described herein. In some embodiments, the host cell stability is increased by greater than 10%, 33%, 50%, 90%, 95% or 99% following administration with any one of the inhibitors described herein. “Stability” as used herein refers to no significant change (e.g., no more than 1%, 2%, 5%, 10%, 15%, 18%, 20%, 25%, 30%, 35% or 40%) in one or more characteristics of a cell over a period of time. The period of time may be at least 1, 2, 3, 4, 5, 6 or 7 weeks, 1 month, or 1, 2, 5, 10, 20, 30, 40, 50, or 60 population doublings of the cell culture. Examples of the characteristics of a cell include growth rate or genome of the cell, expression of endogenous proteins or growth factors by the cell, a heterologous nucleic acid sequence, whether integrated into the genome of the cell, and production of a recombinant protein, for example, with a specific modification, by the cell.

In some embodiments, the inhibitor comprises a small molecule inhibitor. As used herein, “small molecule inhibitor” refers to a small molecule or low molecular weight organic compound that inactivates, inhibits, or antagonizes a target molecule, biomolecule, protein or other biological product.

In some embodiments, the small molecule inhibitor comprises 3-chloro-4-((8-chloro-3-(3-methylbutanoyl)-5-nitro-4-oxo-1,4-dihydroquinolin-2-yl)amino)benzoic acid (Compound 1).

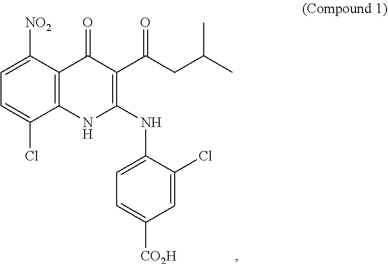 [SMILES: CC(C)CC(=O)c1c(Nc2ccc(C(=O)O)cc2Cl)[nH]c2c(Cl)ccc([N+](=O)[O-])c2c1=O]

In some embodiments, the inhibitor is an antisense molecule, such as a small interfering nucleic acid (siNA). Examples of siNAs include the following: microRNA (miRNA), small interfering RNA (siRNA), double-stranded RNA (dsRNA), and short hairpin RNA (shRNA) molecules. An siNA useful in the invention can be unmodified or chemically-modified. An siNA of the instant invention can be chemically synthesized, expressed from a vector or enzymatically synthesized. Such methods are well known in the art. In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

Other inhibitor molecules that can be used include ribozymes, peptides, DNAzymes, peptide nucleic acids (PNAs), triple helix forming oligonucleotides, and aptamers and modified form(s) thereof directed to sequences in gene(s), RNA transcripts, or proteins.

### Pharmaceutical Compositions

Aspects of the current disclosure relate, in some cases, to a pharmaceutical composition to deliver one or more TLS pathway inhibitors. In some embodiments, the pharmaceutical composition comprises at least one TLS pathway inhibitor and a pharmaceutically acceptable excipient (e.g., carrier). As used herein, “pharmaceutically acceptable excipient” or “pharmaceutically acceptable carrier” refers to a pharmacologically inactive material used together with a pharmacologically active material to formulate the compositions.

The pharmaceutical compositions to be used in the present methods can comprise pharmaceutically acceptable carriers, excipients, or stabilizers in the form of lyophilized formulations or aqueous solutions (Remington, Joseph Price. Remington: the science and practice of pharmacy. Vol. 1. Lippincott Williams & Wilkins, 2006). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the TLS pathway inhibitors which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The TLS pathway inhibitors may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, Joseph Price. Remington: the science and practice of pharmacy. Vol. 1. Lippincott Williams & Wilkins, 2006.

In other examples, the pharmaceutical composition described herein can be formulated in a sustained-release format. Suitable examples of sustained-release preparations include semipermeable matrices of solid hydrophobic polymers containing the TLS pathway inhibitor, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets or capsules, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0.im, particularly 0.1 and 0.5.im, and have a pH in the range of 5.5 to 8.0.

The emulsion compositions can be those prepared by mixing a TLS pathway inhibitor with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the inhibitors as described herein can be aerosolized using a fluorocarbon formulation and a metered dose inhaler, or inhaled as a lyophilized and milled powder.

In some embodiments, the pharmaceutical composition is formulated for oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. The pharmaceutical composition, in some embodiments, is formulated as a solution, emulsion, gel, ointment, cream, suspension, lozenge, tablet, capsule, aerosol, liposome, or lipid nanoparticle.

In some embodiments, the pharmaceutical composition comprises a capsule. In some embodiments, the capsule is administered orally (e.g., ingested). In some embodiments, the capsule or tablet or pill of the pharmaceutical composition is coated or otherwise compounded to afford the advantage of prolonged action. In some embodiments, the tablet, pill, or capsule comprises an inner dosage and an outer dosage component, wherein the outer dosage component forms an envelope over the inner dosage components. Enteric layers or coatings that resist disintegration in the stomach are used, in some embodiments, to separate the inner dosage and the outer dosage to permits the inner dosage to pass through the stomach intact and delay release later in digestion. Enteric layers or coatings can comprise materials such as individual or mixtures of polymeric acids including such materials as shellac, acetyl alcohol, and cellulose acetate.

In some embodiments, the pill or capsule comprises a capsule, wherein said capsule comprises a softgel. In some embodiments, the softgel comprises gelatin. In some embodiments, the gelatin encapsulation of the TLS pathway inhibitor comprises gelatin, glycerin, water, and optionally caramel. In some embodiments, the pills and capsules herein are coated with an enteric coating (e.g., to avoid the acid environment of the stomach, and release most of the lipid agent in the small intestines of a subject). In some embodiments, the enteric coating comprises a polymer barrier that prevents its dissolution or disintegration in the gastric environment, thus allowing the TLS pathway inhibitor (e.g., sulfatides) to reach the small intestines. Examples of enteric coatings include, but are not limited to, Methyl acrylate-methacrylic acid copolymers; Cellulose acetate phthalate (CAP); Cellulose acetate succinate; Hydroxypropyl methyl cellulose phthalate; Hydroxypropyl methyl cellulose acetate succinate (hypromellose acetate succinate); Polyvinyl acetate phthalate (PVAP); Methyl methacrylate-methacrylic acid copolymers; Shellac; Cellulose acetate trimellitate; Sodium alginate; Zein; COLORCON, and an enteric coating aqueous solution (ethylcellulose, medium chain triglycerides [coconut], oleic acid, sodium alginate, stearic acid) (e.g., coated softgels).

In further embodiments, the composition further comprises a solvent (e.g., DMSO).

### Methods of Use

In some embodiments, the compositions of the disclosure are used to treat or prevent one or more viral infection (e.g., RNA virus infection). As used herein, the term “treating” or “treatment” refers to the application or administration of a composition including one or more active agents to a subject, who has a target disease or disorder, a symptom of the disease/disorder, or a predisposition toward the disease/disorder, with the purpose to prevent, cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the virus is a circulating virus (e.g., those presently in the human population or presently in a subset of the human population). In some embodiments, the RNA virus is selected from the group consisting of: coronaviruses, Dengue virus (DENV), Japanese encephalitis virus (JEV), tick-borne encephalitis virus (TBEV), yellow fever virus (YFV), West Nile virus (WNV), Saint Louis encephalitis virus (SLEV), Kunin virus (KUNV), Murray Valley Encephalitis Virus (MVEV), Rocio Virus (ROCV), Simelique Virus (SFV), Powassen Virus (POWV), Mayaro Virus (MAYV), Cosanur Forest Disease Virus (KFDV), Omsk Hemorrhagic Fever Virus (OHFV), Wessels Brown Disease Virus (WDV), Leaping Disease Virus (LIV), Illius Virus (ILHV), Buniamvira Virus (BUNV), Branch Corbera (KOKV), Usutu virus (USUV), Rio Bravo Enncephalitis (RBEV), Negishi Encephalitis (NEGV), Cell Fusing Agent (CFAV) and Kamiti River virus (KRV), Zika virus, and Chikungunya virus. In some embodiments, the RNA virus is selected from the group consisting of: coronavirus, Dengue virus, retrovirus, flavivirus, Nipah virus, West Nile virus, human papillomavirus, respiratory syncytial virus, filovirus, Zaire ebolavirus, Sudan ebolavirus, Marburg virus and influenza virus.

In some embodiments, the virus is Dengue virus (DENV).

In some embodiments, the virus is a coronavirus. In some embodiments, the coronavirus is an alphavirus. Examples of alphacoronaviruses include human coronavirus 229E (HCoV-229E) and human coronavirus NL63 (HCoV-NL63).

In some embodiments, the coronavirus is a betacoronavirus. Examples of betacoronaviruses include: human coronavirus OC43 (HCoV-OC43), human coronavirus HKU1 (HCoV-HKUI), Middle East respiratory syndrome-related coronavirus (MERS-CoV) previously known as novel coronavirus 2012 or HCoV-EMC, severe acute respiratory syndrome coronavirus (SARS-COV) also known as SARS-COV-1 or SARS-classic, and severe acute respiratory syndrome coronavirus (SARS-COV-2) also known as 2019-nCOV or novel coronavirus 2019. In some embodiments, the virus is SARS-CoV-2. In some embodiments, the virus is a variant of SARS-COV-2. Variants of SARS-COV-2 include, but are not limited to, alpha, beta, gamma, delta, omicron BA-1, omicron BA-2, omicron BA-4, omicron BA-5, XBB.1.5, XBB.1.16, and EG.5 strains of SARS-COV-2.

In some embodiments, the subject has long COVID. In some embodiments, the subject has, or is suspected of having long COVID. Symptoms of long COVID include, but are not limited to: cardiomyopathy, neurological issues, diabetes, aging, respiratory system disorders, nervous system and neurocognitive disorders, mental health disorders, metabolic disorders, gastrointestinal disorders, musculoskeletal pain, anemia, headaches, shortness of breath, anosmia, parosmia, muscle weakness, and low fever. In some embodiments, the subject has at least one of the symptoms of long COVID.

The subject, in some embodiments, is a mammal. Mammals include, but are not limited to, farm animals, sport animals, pets, primates, horses, dogs, cats, mice and rats. In some embodiments, the subject is a human. In some embodiments, the subject has, or is suspected of having, at least one RNA viral infection. A subject has, or is suspected of having, an RNA viral infection if the subject has a positive test indicating the presence of one or more antigens or antibodies relating to the virus, was in close contact with someone having the virus, and/or is exhibiting symptoms of the viral infection. A human subject who needs the treatment may be a human patient having, at risk for, or suspected of having a target disease/disorder, such as a SARS infection (e.g., COVID-19).

A subject suspected of having any of such target disease/disorder might show one or more symptoms of the disease/disorder or alternatively may test positive for the infectious agent. A subject at risk for the disease/disorder can be a subject having one or more of the risk factors for that disease/disorder or exposed to the infectious agent.

In some embodiments, the compositions described herein are administered to a subject in an effective amount, that is, an amount sufficient to inhibit or reduce the activity of TLS polymerases by at least 20% (e.g., 30%, 40%, 50%, 60%, 70%, 80%, 90% or greater) in vivo. As used herein, “an effective amount” refers to the amount of each active agent required to confer therapeutic effect on the subject, either alone or in combination with one or more other active agents. Determination of an amount of the composition required to achieve the therapeutic effect would be evident to one of skill in the art. Effective amounts vary depending on the condition being treated, the severity of the condition, individual patient parameters including age, physical condition, size, gender and weight, the duration of the treatment, the nature of concurrent therapy (if any), the specific route of administration and like factors. These factors are well known to those of ordinary skill in the art and can be addressed with no more than routine experimentation.

In some embodiments, the appropriate dosage of a TLS pathway inhibitor described herein will depend on the specific TLS pathway inhibitor, TLS pathway inhibitors, and/or other therapeutic agents (or compositions thereof) employed, as well as the type and severity of the disease/disorder, whether the TLS pathway inhibitor is administered for preventive or therapeutic purposes, previous therapy, the patient's clinical history and response to the antagonist, and the discretion of the attending physician. Methods of determining whether a dosage resulted in the desired result would be evident to one of skill in the art. In some embodiments, the desired result is a reduction in viral load (e.g., number of viral copies per unit blood or plasma).

Administration of one or more TLS pathway inhibitors can be continuous or intermittent, depending, for example, upon the subject's physiological condition, whether the purpose of the administration is therapeutic or prophylactic, and other factors known to skilled practitioners. The administration of a TLS pathway inhibitor may be essentially continuous over a preselected period of time or may be in a series of spaced dose, e.g., either before, during, or after developing a target disease or disorder.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some examples, the pharmaceutical composition is administered intravenously. In some embodiments, the pharmaceutical composition is administered orally.

The pharmaceutical compositions described herein may be given as an individual dose, but are not restricted to one dose. The administration can comprise two, three, four, five, six, seven, eight, nine, 10 or more, administrations of the pharmaceutical composition. Where more than one administration is required to achieve a therapeutic effect in response to the pharmaceutical composition, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. The administrations can also be spaced by time intervals of one day, two days, three days, four days, five days, six days, seven days or more days.

The particular dosage regimen, i.e., dose, timing and repetition, used in the method described herein will depend on the particular subject and that subject's medical history.

In one example, dosages for a TLS pathway inhibitor as described herein may be determined empirically in individuals who have been given one or more administration(s) of the TLS pathway inhibitor.

Generally, for administration of any of the TLS pathway inhibitors described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the TLS pathway inhibitor, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the TLS pathway inhibitor used) can vary over time.

In some embodiments, for an adult patient of normal weight, doses ranging from about 0.3 to 5.00 mg/kg may be administered. In some examples, the dosage of the TLS pathway inhibitor described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

In some embodiments, more than one composition, or a combination of a composition described herein and another suitable therapeutic agent, may be administered to a subject in need of the treatment. The composition described herein can also be used in conjunction with other agents that serve to enhance and/or complement the effectiveness of the agents.

In some embodiments, the compositions described herein may be administered with another suitable therapeutic agent to a subject in need of the treatment. The composition described herein can be used in prior to administration, after administration, or in conjunction with other agents that serve to enhance and/or complement the effectiveness of the composition and the agents. When the composition is co-administered with an additional therapeutic agent, suitable therapeutically effective dosages for each agent may be lowered due to the additive action or synergy. Examples of secondary suitable therapeutic agents for subjects having SARS-COV-2 or long COVID include anti-viral agents, such as remdesivir, β-D-N⁴-hydroxycytidine, convalescent plasma, COVID-19 monoclonal antibodies, and favipiravir. Any of the compositions described herein may be utilized in conjunction with other types of therapy for viral infections including other types of therapy for downstream effects of viral infections such as rest, fluids, and pain medication. Such therapies can be administered with the composition simultaneously or sequentially, before or after the composition, as determined by medical expert.

When co-administered with an additional therapeutic agent, suitable therapeutically effective dosages for each agent may be lowered due to the additive action or synergy.

### Kits

The present disclosure also provides kits for use in treating or alleviating RNA viral infections (e.g., SARS, COVID-19). Such kits can include one or more containers comprising a TLS pathway inhibitor, e.g., any of those described herein.

In some embodiments, the kit can comprise instructions for use in accordance with any of the methods described herein. The included instructions can comprise a description of administration of the TLS pathway inhibitor, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering a TLS pathway inhibitor to an individual at risk of the target disease.

The instructions relating to the use of a TLS pathway inhibitor generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating an RNA virus (e.g., COVID-19). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a TLS pathway inhibitor as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. SARS-COV-2 Triggers Genome Instability In Vitro and In Vivo

To test whether SARS-COV-2 triggers genome instability, relative transcript levels of the DNA damage response (DDR), DNA repair, and translesion synthesis (TLS) genes were quantified at 48 hours post-SARS-COV2 infection in A549-ACE2+ cells using qRT-PCR (n=9). In vitro SARS-COV2 infection increased expression of DDR genes (ATM, ATR, including CHK1), DNA repair genes from double-strand break repair (DSBR: BRCA1, MRE11A, PARP1, and RAD51) and nucleotide excision repair (NER: XPA) pathways, and mutagenic TLS genes (POLh, POLK, POLi, REV1, and REV7). Similarly, ATR expression was detected in the lung tissue of the Golden Syrian hamster at 30 days post-SARS-COV-2 infection.

At the protein level, each factor exhibited a unique pattern of upregulation. For example, SARS-COV-2 infection increased protein expression of REV1 and REV7 in A549-ACE2+ cells, with peak expression levels between 4 to 8 hours post-infection (n=3). This unique expression pattern of TLS genes was not observed in influenza A virus-infected A549-ACE2+ cells, where a different set of DDR genes (DDB2, DDB1, DDIT4, SMC5) were upregulated. Immunohistochemical analysis of human autopsy COVID-19 lung tissues showed an increased expression of gH2AX compared to post-mortem interval (PMI)-matched controls. This was also observed in lung tissue of Golden Syrian hamster up to 30 days post-SARS-COV-2 infection. 53BP1, an important transducer of DNA damage and genome instability, was highly expressed in the terminal bronchioles, but the overall expression in the surrounding lung tissue was less pronounced.

Within a limited group of patients investigated at least three months following acute COVID, longitudinal expression of 53BP1 at three intervals six months apart following the first visit showed a significant decrease in expression in three of the five patients, suggesting that SARS-COV-2 infection modulates the expression of genome instability markers in cells, autopsy lung tissues, Golden Syrian hamster lung tissue, and sera from post-COVID patients.

Telomeric dysfunction, a marker of genomic instability, was examined in A549-ACE2+ cells following SARS-COV-2 infection. Significant telomere instability-marked by a reduction and lengthening of telomeres—was found in autopsy patient lung tissues, infected A549-ACE2+ cells, and lung tissue of Golden Syrian hamster for 30 days post-SARS-COV-2 infection. Further, expression of the two shelterin proteins, TRF2 and POT1, which encapsulate telomeres into protective units, was significantly repressed in autopsy lung tissues and infected cells, in contrast to the elevated hTERT expression in infected A549-ACE2+ cells and the lung tissue of Golden Syrian hamster 30 days post-SARS-COV-2 infection. As different cell lines exhibited distinct telomere lengths, SARS-COV-2 may be impacting the telomere biology uniquely in different tissues.

### Example 2. Inhibition of the TLS Pathway

Since SARS-COV-2 increases the expression of mutagenic TLS polymerases, a two-fold hypothesis was tested: a) whether SARS-COV-2-dependent increased TLS expression inadvertently causes host cell genetic alterations, and b) whether inhibiting the TLS pathway diminishes the deleterious consequences of SARS-COV-2 infection. A general increase in the mutation burden in infected cells was observed, as a 120% increase in mutation frequency at the HPRT (hypoxanthine phosphoribosyltransferase) gene was observed in A549-ACE2+ cells infected with SARS-COV-2. Likewise, other mutability events, such as microsatellite instability (MSI), where insertions or deletions occur at a high frequency at repetitive DNA, were high not only in A549-ACE2+ infected cells but also in most of the autopsy lung tissues compared to the PMI-matched controls. Furthermore, a significant reduction in expression of the mismatch repair (MMR) proteins, MSH2, MLH1 and MSH6 was observed in A549-ACE2+ cells infected with SARS-COV-2. To determine MMR status in patients post-COVID, the longitudinal expression of MSH2 protein in patient sera was tested and found to be significantly reduced in two of the five tested patients.

To determine whether TLS inhibition might suppress the noted mutagenic events, a TLS inhibitor (Compound 1) that specifically targets the REV7 interface of REV1 TLS polymerase, was tested to determine whether it suppresses genetic alterations in host cell DNA. Compound 1 treatment was found to suppress both the SARS-COV-2-dependent HPRT mutagenesis and MSI in infected A549-ACE2+ cells, suggesting that increased expression of TLS polymerases contributes to the elevation of mutagenic events and that therapeutic inhibition of TLS can suppress SARS-COV-2-dependent deleterious consequences.

Next, whether other genome instability markers were also repressed by the Compound 1 treatment in SARS-COV-2 infected cells was examined. Compound 1 treatment of the A549-ACE2+ cells suppressed transcript expression of all the DDR, TLS, and DNA repair genes. Likewise, the enhanced expression of gH2AX in SARS-CoV-2 infected A549-ACE2+ cells at 48 hours was suppressed by up to 40-fold post-Compound 1 treatment. Compound 1 treatment did not rescue telomere instability in SARS-CoV-2 infected A549-ACE2+ cells; however, suggesting that SARS-COV-2 may impact telomere instability by an independent pathway.

It was also observed that the Compound 1 was also able to directly suppress the proliferation of SARS-COV-2 in three independent cell lines-Vero, A549-ACE2+, and Calu-3 cells, as noted by the relative mRNA content in cells. This result was also observed in the STATIKO cell line, suggesting independence from the immune pathway and a possible role of REV1 in SARS-COV-2 propagation. Because siREV1 knockdown in A549-ACE2+ cells also suppressed SARS-COV-2 propagation, it is thought that REV1 has a specific role in virus propagation in cells. As REV1 inhibition was recently shown to trigger autophagy, whether Compound 1 treatment induces autophagy to limit SARS-COV-2 was examined. On its own, SARS-COV-2 infection steadily increases LC3 expression over time, without an increase in p62; however, Compound 1 treatment significantly increases the expression of p62 and LC3 in SARS-COV-2 infected cells, indicating that Compound 1 treatment upregulates p62 expression that may promote lysosomal degradation of SARS-COV-2, limiting its propagation in cells. With respect to RNA sequencing data, a gene enrichment for viral myocarditis in the REV1KO mouse embryonic fibroblasts was observed. Due to that observation, whether Compound 1 treatment might suppress one of the factors, CASP9, involved in SARS-COV-2-dependent increase in myocarditis, was tested. Treatment of cells with Compound 1 was found to CASP9 expression, suggesting mechanisms of genome instability might associate with myocarditis with therapeutic implications during long COVID.

### Example 3. Reduction of mRNA and Viral Nucleocapsids in A549-ACE2+ Cells Infected with Different SARS-COV-2 Variants

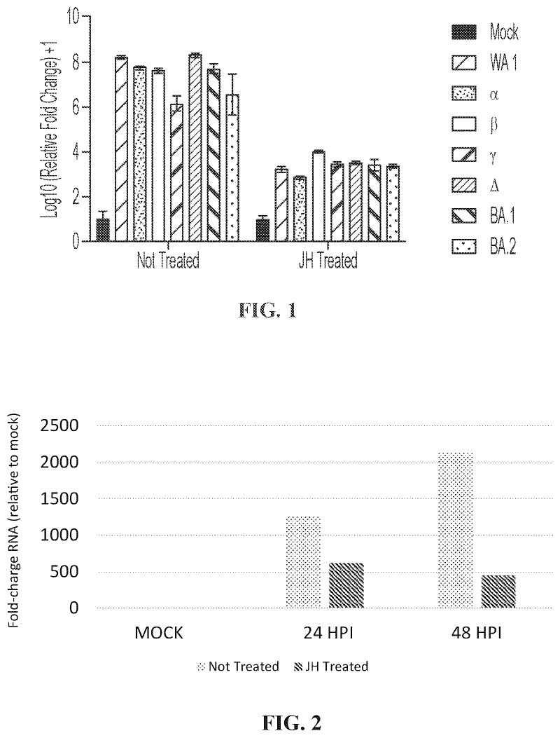

A549-ACE2+ cells were infected with SARS-COV-2 variants (alpha, beta, gamma, delta, BA.1 (omicron), or BA.2 (omicron)), USA-WA1/2020 (SARS-COV-2; “WA”), or mock-infected. Cells were then treated with Compound 1 (10 μM) or untreated. Forty-eight hours later, viral mRNA levels and the percentage of cells positive for viral nucleocapsids were measured. The results demonstrate that Compound 1 reduces viral mRNA levels (FIG. 1) and the percentage of viral nucleocapsid-positive cells (FIG. 4) across all variants and the USA-WA1/2020 strain compared to the untreated groups.

### Example 4. Compound 1 and Dengue Virus

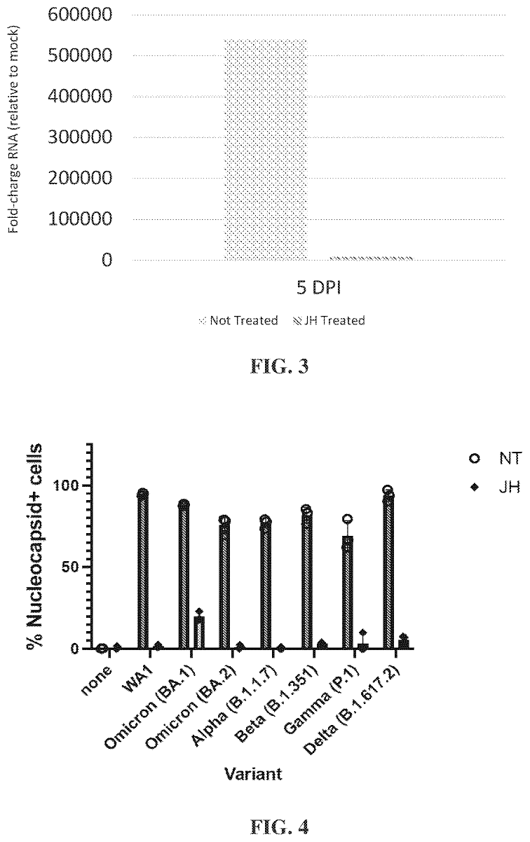

Compound 1 (10 μM) was administered to A549-ACE2+ cells infected with Dengue virus and the resulting viral loads were measured (FIGS. 2 and 3). Compared to non-treated cells, those treated with Compound 1 suppressed Dengue virus by 77-fold. Data is shown 24 hours, 48 hours, and 5 days post-infection (PI).

### REFERENCES

1. Taquet, M.; Dercon, Q.; Luciano, S.; Geddes, J. R.; Husain, M.; Harrison, P. J., Incidence, co-occurrence, and evolution of long-COVID features: A 6-month retrospective cohort study of 273,618 survivors of COVID-19. PLOS Med 2021, 18 (9), e1003773.
2. Douaud, G.; Lee, S.; Alfaro-Almagro, F.; Arthofer, C.; Wang, C.; Mccarthy, P.; Lange, F.; Andersson, J. L. R.; Griffanti, L.; Duff, E.; Jbabdi, S.; Taschler, B.; Keating, P.; Winkler, A. M.; Collins, R.; Matthews, P. M.; Allen, N.; Miller, K. L.; Nichols, T. E.; Smith, S. M., SARS-COV-2 is associated with changes in brain structure in UK Biobank. Nature 2022.
3. Xie, Y.; Xu, E.; Bowe, B.; Al-Aly, Z., Long-term cardiovascular outcomes of COVID-19. Nat Med 2022, 28 (3), 583-590.
4. Mavrikaki, M.; Lee, J. D.; Solomon, I. H.; Slack, F. J., Severe COVID-19 induces molecular signatures of aging in the human brain. medRxiv 2021.
5. Lee, S.; Yu, Y.; Trimpert, J.; Benthani, F.; Mairhofer, M.; Richter-Pechanska, P.; Wyler, E.; Belenki, D.; Kaltenbrunner, S.; Pammer, M.; Kausche, L.; Firsching, T. C.; Dietert, K.; Schotsaert, M.; Martinez-Romero, C.; Singh, G.; Kunz, S.; Niemeyer, D.; Ghanem, R.; Salzer, H. J. F.; Paar, C.; Mulleder, M.; Uccellini, M.; Michaelis, E. G.; Khan, A.; Lau, A.; Schonlein, M.; Habringer, A.; Tomasits, J.; Adler, J. M.; Kimeswenger, S.; Gruber, A. D.; Hoetzenecker, W.; Steinkellner, H.; Purfurst, B.; Motz, R.; Di Pierro, F.; Lamprecht, B.; Osterrieder, N.; Landthaler, M.; Drosten, C.; Garcia-Sastre, A.; Langer, R.; Ralser, M.; Eils, R.; Reimann, M.; Fan, D. N. Y.; Schmitt, C. A., Virus-induced senescence is a driver and therapeutic target in COVID-19. Nature 2021, 599 (7884), 283-289.
6. Victor, J.; Deutsch, J.; Whitaker, A.; Lamkin, E. N.; March, A.; Zhou, P.; Botten, J. W.; Chatterjee, N., SARS-COV-2 triggers DNA damage response in Vero E6 cells. Biochem Biophys Res Commun 2021, 579, 141-145.
7. Chatterjee, N.; Walker, G. C., Mechanisms of DNA damage, repair, and mutagenesis. Environ Mol Mutagen 2017, 58 (5), 235-263.
8. Yamanaka, K.; Chatterjee, N.; Hemann, M. T.; Walker, G. C., Inhibition of mutagenic translesion synthesis: A possible strategy for improving chemotherapy? PLOS Genet 2017, 13 (8), e1006842.
9. Chatterjee, N.; Whitman, M. A.; Harris, C. A.; Min, S. M.; Jonas, O.; Lien, E. C.; Luengo, A.; Vander Heiden, M. G.; Hong, J.; Zhou, P.; Hemann, M. T.; Walker, G. C., REV1 inhibitor JH-RE-06 enhances tumor cell response to chemotherapy by triggering senescence hallmarks. Proc Natl Acad Sci USA 2020, 117 (46), 28918-28921.
10. Ikeh, K. E.; Lamkin, E. N.; Crompton, A.; Deutsch, J.; Fisher, K. J.; Gray, M.; Argyle, D. J.; Lim, W. Y.; Korzhnev, D. M.; Hadden, M. K.; Hong, J.; Zhou, P.; Chatterjee, N., REV1 Inhibition Enhances Radioresistance and Autophagy. Cancers (Basel) 2021, 13 (21).
11. Nilsson-Payant, B. E.; Uhl, S.; Grimont, A.; Doane, A. S.; Cohen, P.; Patel, R. S.; Higgins, C. A.; Acklin, J. A.; Bram, Y.; Chandar, V.; Blanco-Melo, D.; Panis, M.; Lim, J. K.; Elemento, O.; Schwartz, R. E.; Rosenberg, B. R.; Chandwani, R.; tenOever, B. R., The NF-kappaB Transcriptional Footprint Is Essential for SARS-COV-2 Replication. J Virol 2021, 95 (23), e0125721.
12. Fink, L. S.; Roell, M.; Caiazza, E.; Lerner, C.; Stamato, T.; Hrelia, S.; Lorenzini, A.; Sell, C., 53BP1 contributes to a robust genomic stability in human fibroblasts. Aging (Albany NY) 2011, 3 (9), 836-45.
13. Feldser, D. M.; Hackett, J. A.; Greider, C. W., Telomere dysfunction and the initiation of genome instability. Nat Rev Cancer 2003, 3 (8), 623-7.
14. Baudrin, L. G.; Deleuze, J. F.; How-Kit, A., Molecular and Computational Methods for the Detection of Microsatellite Instability in Cancer. Front Oncol 2018, 8, 621.
15. Wojtaszek, J. L.; Chatterjee, N.; Najeeb, J.; Ramos, A.; Lee, M.; Bian, K.; Xue, J. Y.; Fenton, B. A.; Park, H.; Li, D.; Hemann, M. T.; Hong, J.; Walker, G. C.; Zhou, P., A Small Molecule Targeting Mutagenic Translesion Synthesis Improves Chemotherapy. Cell 2019, 178 (1), 152-159 e11.
16. Choi, Y.; Bowman, J. W.; Jung, J. U., Autophagy during viral infection—a double-edged sword. Nat Rev Microbiol 2018, 16 (6), 341-354.
17. Jansen, J. G.; Langerak, P.; Tsaalbi-Shtylik, A.; van den Berk, P.; Jacobs, H.; de Wind, N., Strand-biased defect in C/G transversions in hypermutating immunoglobulin genes in Rev1-deficient mice. J Exp Med 2006, 203 (2), 319-23.
18. Lee, A. C.; Castaneda, G.; Li, W. T.; Chen, C.; Shende, N.; Chakladar, J.; Taub, P. R.; Chang, E. Y.; Ongkeko, W. M., COVID-19 Severity Potentially Modulated by Cardiovascular-Disease-Associated Immune Dysregulation. Viruses 2021, 13 (6).
19. Mesri, E. A.; Feitelson, M. A.; Munger, K., Human viral oncogenesis: a cancer hallmarks analysis. Cell Host Microbe 2014, 15 (3), 266-82.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a viral infection in a subject, wherein the method comprises administering to the subject an inhibitor of the translesion synthesis (TLS) pathway.

2. The method of claim 1, wherein the inhibitor comprises an inhibitor of a mutagenic translesion synthesis polymerase.

3. The method of claim 2, wherein the mutagenic translesion synthesis polymerase is selected from the group consisting of: POLh, POLK, POLI, REV1, REV3L, and REV7.

4. The method of claim 3, wherein the mutagenic translesion synthesis polymerase comprises REV1.

5. The method of any one of claims 1-4, wherein the inhibitor of a translesion synthesis polymerase comprises a small molecule inhibitor.

6. The method of claim 5, wherein the small molecule inhibitor comprises Compound 1:

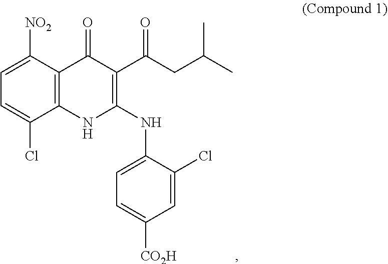 [SMILES: CC(C)CC(=O)c1c(Nc2ccc(C(=O)O)cc2Cl)[nH]c2c(Cl)ccc([N+](=O)[O-])c2c1=O]

7. The method of any one of the preceding claims, wherein the viral infection is caused by an RNA virus.

8. The method of claim 7, wherein the RNA virus is selected from the group consisting of: coronavirus, Dengue virus, retrovirus, flavivirus, Nipah virus, West Nile virus, human papillomavirus, respiratory syncytial virus, filovirus, Zaire ebolavirus, Sudan ebolavirus, Marburg virus, and influenza virus.

9. The method of claim 8, wherein the viral infection is coronavirus.

10. The method of claim 9, wherein the coronavirus comprises a betacoronavirus.

11. The method of claim 10, wherein the betacoronavirus comprises SARS-COV-2 or a variant thereof.

12. The method of claim 11, wherein the SARS-COV-2 variant is selected from the group consisting of: alpha, beta, gamma, delta, omicron BA-1, omicron BA-2, omicron BA.4, omicron BA.5, XBB.1.5, XBB.1.16, and EG.5.

13. The method of claim 11, wherein the variant comprises a circulating SARS-COV-2 variant.

14. The method of claim 1, wherein the subject has, or is suspected of having, long COVID.

15. The method of claim 14, wherein the subject is human.

16. The method in claim 15, wherein the subject has at least one of: cardiomyopathy, neurological issues, diabetes, respiratory system disorders, nervous system and neurocognitive disorders, mental health disorders, metabolic disorders, gastrointestinal disorders, musculoskeletal pain, anemia, headaches, shortness of breath, anosmia, parosmia, muscle weakness, and low fever.

17. The method of any of the preceding claims, wherein the subject is administered the inhibitor by oral administration or intravenous administration.

18. A pharmaceutical composition suitable for treating a viral infection in a subject, wherein the pharmaceutical composition comprises an inhibitor of the translesion synthesis (TLS) pathway.

19. The pharmaceutical composition of claim 18, wherein the pharmaceutical composition further comprises a pharmaceutically acceptable carrier.

20. The pharmaceutical composition of any one of claims 18-19, wherein the inhibitor of the TLS pathway comprises an inhibitor of a mutagenic translesion synthesis polymerase.

21. The pharmaceutical composition of claim 20, wherein the mutagenic translesion synthesis polymerase is selected from the group consisting of: POLh, POLK, POLi, REV1, or REV7.

22. The pharmaceutical composition of claim 21, wherein the mutagenic translesion synthesis polymerase comprises REV1.

23. The composition of any one of claims 18-22, wherein the inhibitor of the TLS pathway comprises a small molecule inhibitor.

24. The pharmaceutical composition of claim 23, wherein the small molecule inhibitor comprises Compound 1.

25. The pharmaceutical composition of any one of claims 18-24, wherein the viral infection is caused by an RNA virus.

26. The pharmaceutical composition of claim 25, wherein the RNA virus is selected from the group consisting of: coronavirus, Dengue virus, retrovirus, flavivirus, Nipah virus, West Nile virus, human papillomavirus, respiratory syncytial virus, filovirus, Zaire ebolavirus, Sudan ebolavirus, Marburg virus, and influenza virus.

27. The pharmaceutical composition of claim 26, wherein the virus is a coronavirus.

28. The pharmaceutical composition of claim 27, wherein the coronavirus is a betacoronavirus.

29. The pharmaceutical composition of claim 28, wherein the betacoronavirus comprises SARS-COV-2 or a variant thereof.

30. The pharmaceutical composition of claim 29, wherein the SARS-COV-2 variant is selected from the group consisting of: alpha, beta, gamma, delta, omicron BA-1, omicron BA-2, omicron BA.4, omicron BA.5, XBB.1.5, XBB.1.16, and EG.5.

31. The pharmaceutical composition of claim 30, wherein the variant comprises a circulating SARS-COV-2 variant.

32. The pharmaceutical composition of any one of claims 18-31, wherein the subject has or is suspected of having long-COVID.

33. The pharmaceutical composition of claim 32, wherein the subject is human.

34. The pharmaceutical composition of 33, wherein the subject has at least one of: cardiomyopathy, neurological issues, diabetes, aging, respiratory system disorders, nervous system and neurocognitive disorders, mental health disorders, metabolic disorders, gastrointestinal disorders, musculoskeletal pain, anemia, headaches, shortness of breath, anosmia, parosmia, muscle weakness, and low fever.

35. The pharmaceutical composition of any one of claims 18-34, wherein the pharmaceutical composition is administered by oral administration or intravenous administration.

36. The pharmaceutical composition of any one of claims 18-35, wherein the pharmaceutical composition is formulated as a capsule.

37. The pharmaceutical composition of claim 36, wherein the pharmaceutical composition is acceptable for oral administration.

38. The pharmaceutical composition of any of claims 34-37, wherein the pharmaceutical composition is administered according to a dosing schedule sufficient to alleviate at least one symptom.
